(19) United States
(12) Patent Application Publication
(10) Pub. No.: US 2026/0272332 A1
(43) Pub. Date: Sep. 17, 2026

(54) WEARABLE BIOSENSOR WITH BIOMETRIC ID SYSTEM

(71) Applicant: AQUILX INCORPORATED, Bonsall, CA (US)

(72) Inventors: Farshad TEHRANI, Bonsall, CA (US); Hazhir TEYMOURIAN, San Diego, CA (US); Brian WUERSTLE, Pacifica, CA (US)

(21) Appl. No.: 19/168,286

(22) PCT Filed: Mar. 25, 2024
(86) PCT No.: PCT/US2024/021347

§ 371 Date: Sep. 23, 2025

Related U.S. Application Data
(60) Provisional application No. 63/454,454, filed on Mar. 24, 2023.

(51) Int. Cl. A61B 5/117; A61B 5/00; A61B 5/145; A61B 5/1468

(52) U.S. Cl. CPC A61B 5/117; A61B 5/0004; A61B 5/14514; A61B 5/1468; A61B 5/685

(57) ABSTRACT

A system for measuring target analytes in a wearer including a wearable biosensor device and biometric identity verification systems for recognition of the wearer's electrochemical signature, fingerprints, voice, gait, face, iris, retina and the like. A single biometric identity verification system may be used, or a multi-modal version.

### STATEMENT OF PRIORITY AND RELATION TO OTHER APPLICATIONS

This application claims priority to, and the full benefit of, U.S. provisional patent application Ser. No. 63/454,454 filed Mar. 24, 2023 and U.S. nonprovisional patent application Ser. No. 18/586,283 (the '283 application) filed Feb. 23, 2024, which is incorporated herein by reference. This application also claims priority to U.S. nonprovisional application Ser. No. 17/513,424 filed Nov. 17, 2023; U.S. nonprovisional application Ser. No. 18/528,763 filed Dec. 4, 2023 and international patent application number PCT/US24/10775 filed Jan. 8, 2024.

### FIELD OF THE INVENTION

The field of the invention is a system incorporating a wearable biosensor device for measuring target analytes in a wearer with subsystems for verifying identity of a wearer of the biosensor device.

### INITIAL ASPECTS OF THE INVENTION

The invention is a system incorporating a wearable biosensor device for measuring target analytes in a wearer with subsystems for verifying identity of a wearer of the biosensor device. The system comprises a biometric identity verification subsystem (sometimes herein “biometric ID subsystem”) which in some embodiments comprises multiple biometric modalities for identity verification (sometimes herein “multi-modal ID”). Biometric ID or multi-modal ID subsystems can be incorporated into any wearable device capable of sampling target analytes in a living body of a wearer. Various embodiments of the wearable biosensor without biometric ID or multi-modal ID are disclosed in the '283 application and incorporated herein. The present invention's biometric ID subsystems are novel improvements to the application of the '283 invention.

### BRIEF DESCRIPTION OF THE FIGURES

FIG. 1 contains perspective views of the skin side (right) and back side (left) of the wearable biosensor device.

FIG. 2 contains illustrative diagrams of slopes of metabolic analytes and skin temperature collected by the wearable biosensor and icons for software modules of the system.

FIG. 3 is a drawing of one embodiment of an electrochemical signature for glucose as the target analyte.

FIG. 4 (FIG. 2 of the '283 application) is an exploded view of one embodiment of the wearable biosensor device with disposable and reusable modules having a recessed cover, and the electrochemical subsystem.

FIG. 5 (FIG. 3 of the '283 application) is an exploded view of another embodiment of the device with disposable and reusable modules having a basic cover, and the electrochemical subsystem.

FIG. 6A is an exploded view of an embodiment of the device with disposable and reusable modules with an elastic insulative base, and FIG. 6B is the same but without the elastic insulative base, and with the electrochemical subsystem. These are

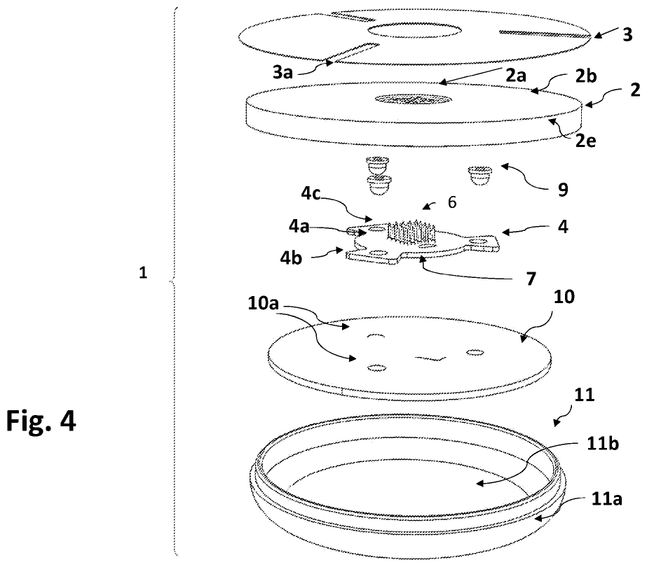

FIGS. 4A-4B of the '283 application, respectively.

### EXAMPLES OF USE CASES

There are a number of situations in which verifying the identity of a user of a wearable device is necessary.

An important example is monitoring of substance abuse in individuals with a history of alcohol or drug addiction, either by law enforcement officials or by clinical personnel in substance abuse programs. It is important to verify that a certain person is wearing the monitor. For example, ID verification ensures accurate and secure tracking of substance use for addiction management, including various classes of drugs such as opioids, alcohol, stimulants, depressants, hallucinogens, and other substances. In various settings, authorities (government or private) need to monitor substance abuse in subjects, a portion of whom might attempt to falsify findings by having another person wear the substance monitor, thus passing off acceptable results for what would otherwise be unacceptable.

Traditional methods of substance monitoring, such as random or scheduled drug testing, can be time-consuming without providing accurate or timely information regarding substance use. For example, in a large metropolitan area, round trip travel to a designated location for biometric readings can require a person to leave a place of employment and take several hours. Prior to the invention herein, methods for sampling target analytes either have required residential care or time-consuming day trips to check blood levels, or have been susceptible to tampering or deception by individuals attempting to evade detection. Consequently, there is a need for a more secure, reliable, and continuous method of monitoring various substances of abuse in individuals. The invention provides a reliable way to verify the wearer's ID and in so doing can reduce the cost of substance abuse programs by shortening the time needed for residential stays. The wearable sensor, for example, is capable of continuously monitoring various substances, including different classes of drugs such as opioids, alcohol, stimulants, depressants, hallucinogens, and others, in the interstitial fluid (ISF) and detecting their presence in near real-time.

Other important applications for the present invention include those involving security clearances for access to facilities or to online resources. If used properly and monitored by security authorities, the novel combination of biometric systems herein insures accurate ID of wearers of devices.

Athletic trainers and administrators for sports teams can also use the invention profitably. For example, managers of professional sports teams can insure remotely that their team's investment in players is safeguarded by remote monitoring of a player's compliance with exercise regimens, or other health and fitness, without having to provide residential living, say, in a training camp. Or remote monitoring with the present invention in the off-season allows players to live and train anywhere they have internet access, but also to be tracked by team staff.

### Wearable Biosensor Devices

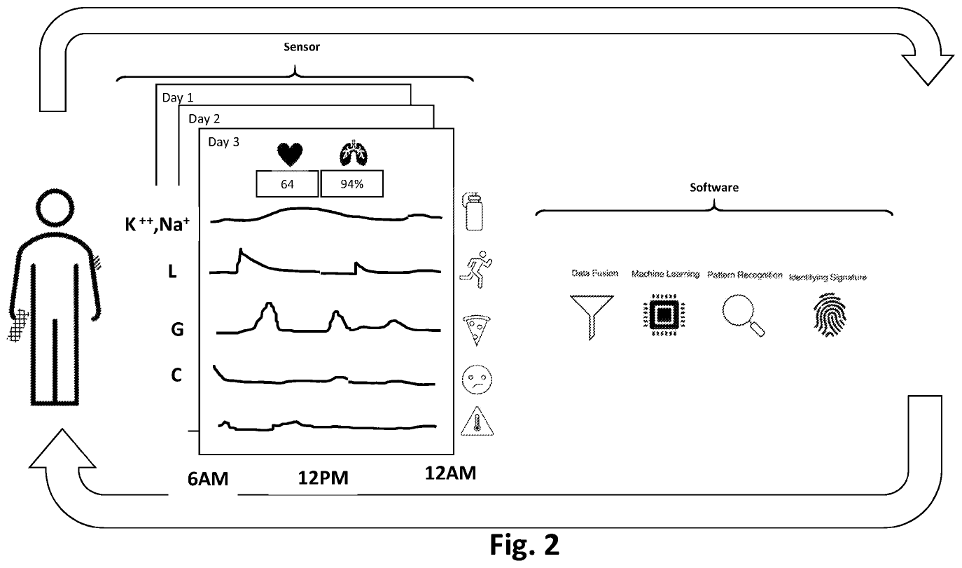

As disclosed further in the '283 application incorporated herein, the wearable biosensor with multi-modal ID herein comprises a microneedle array in which microneedles are integral with a substrate, composed of a nonconductive material. A nonconductive cover with holes corresponding to the microneedles is placed on top of the microneedle array and then the microneedle array and cover are sealed together with curable resin for strength and a liquid-tight seal. The microneedles are first coated with a conductive layer (metallic or conductive polymer) by a PVD or plating process. Various chemical layers are then added on top of the conductive layer, including at least one biorecognition layer (included in the chemical layers), and the biorecognition layer's enzymes react with target analytes in the ISF and generate electrical signals transmitted by the conductive layer to an electronics unit. All of the foregoing is housed in the wearable biosensor. FIGS. 4, 5, 6A and 6B, discussed elsewhere herein, depict various embodiments of the wearable biosensor (FIGS. 2, 3, 4A-B from the '283 application) without biometric ID subsystems. The multi-modal ID subsystems are built into this wearable biosensor, as shown herein. The reference numbers in FIGS. 4, 5, 6A and 6B are the same as in the '283 application.

### Additional Aspects of the Invention

The biometric ID subsystems herein detect a unique and reliable user biometric signature that ensures the wearer's identity and prevents tampering or manipulation of the monitoring process.

The biometric ID subsystems for detecting and processing biometric signatures herein are selected from the group consisting of electrochemical, voice, fingerprint, gait, face, iris, and retina. Other such biometric ID subsystems fit within this group as long as they can be implemented by means of sensors which can be built into a wearable biosensor.

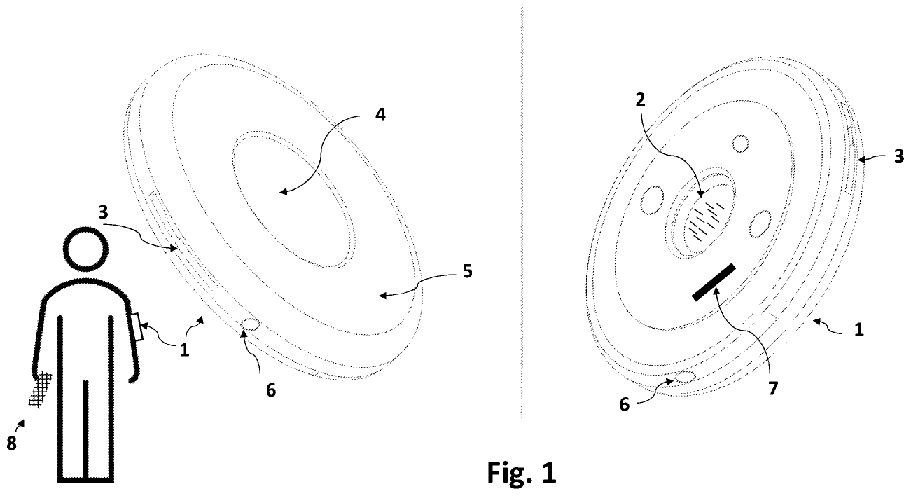

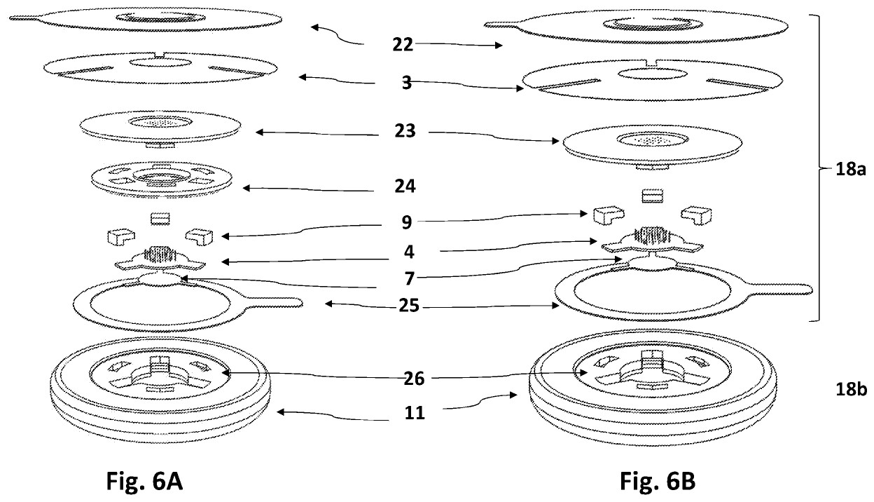

FIG. 1 is an embodiment of the backside and front side (shown with microneedles) of a continuous monitoring wearable device 1 configured to be placed on the upper arm or (other convenient location) of a wearer of an integrated multi-modal ID verification system. The wearable biosensor device is wirelessly connected to an external computing device, here a smart mobile device 8 held by the wearer. FIG. 1 shows locations for electrochemical sensors 2 (microneedles), voice recognition 3 (microphone), touch 4 (fingerprint sensor), gait analysis 5 (magnetometer, accelerometer, gyroscope, GPS), iris, retina or facial recognition 6 (optical sensor and camera) and force touch 7 (strain/pressure sensor) as well as temperature fluctuation recognition (temperature sensor, e.g., conductive trace in FIG. 6 of the '283 application). Various other embodiments of the system use at least one or more of these modalities and biometric ID subsystems.

At least one of the biometric ID subsystems is incorporated into a wearable device (such as e.g., in the '283 application and other devices described herein) having an electrochemical sensor electrode (microneedles) connected to an electronics unit (e.g., a printed circuit board (PCB) or application specific integrated circuit (ASIC)) with electrical connections. In various embodiments, the wearable biosensor device includes electrical connections to one or more of a microphone for capturing the user's voice input, a fingerprint sensor for capturing the user's fingerprint, a micro-sized camera for capturing facial and iris features, monitors for a gait analysis module, an electrochemical multiplexed bio-analyzer, and other recognition elements for verifying the wearer's identity.

The present invention comprises software comprising a database for storing information gathered by the wearable biosensor device. The database runs on a software platform in the cloud and can be accessible in an app running on an external computing device (e.g., smart mobile device) connected wirelessly to the wearable biosensor device, or can be located solely on the smart mobile device.

All of the biometric ID subsystems herein can be combined with any one or more of the other biometric modalities to improve overall system performance and security. Multi-modal ID can help mitigate the limitations of individual biometric techniques and provide more reliable user authentication.

Initial Biometric Signature. A desirable way to implement the system is to implant the microneedles of the wearable biosensor device in the presence of a clinician, law enforcement official or other administrator so that the identity of the wearer is verified in person. During this initial identity session, at least one initial biometric signature is taken in one or more biometric modalities so that identity of the wearer is certain, and is retained in the system database for generating a comparison when later biometric signatures are recorded by the system. This information is stored in an account for the wearer kept by the clinician or other administrator in charge of the monitoring program.

Later Biometric Signature. After the initial identity session, the wearer's identity is verified by the system by taking later biometric signatures which are then compared in the system to the initial biometric signature. The timing and sequence of these later biometric signatures are dictated automatically within the system, or they may be managed by an administrator given access to interact with the wearer's account within the system. In various embodiments the invention herein incorporates techniques for reducing deception such as randomly-timed requests marked by, say, a chime and notification on the smart mobile device that the wearer provide input for one or more of the biometric sensors. For example, the notification can be “Look first into the optical sensor and then apply your index finger to the touch sensor” within a short time such as 30 or 60 seconds, and require a limited number repeated cycles with different combinations until successful. The app can be configured so that the wearer's inability to perform successful verification after these prompts is reported to a system administrator. Sound notifications but being balanced armature (BA) speakers and can be tuned for specific audio profiles, for wearables focusing on high-quality audio output.

Very Low Data Episode. If the microneedles are removed from the wearer's skin, one possibility is that the initial wearer is attempting to substitute another person illicitly. The system tracks whether the wearable biosensor device is removed from contact with bodily fluid (ISF or blood). Removal of the device is reported to the software platform within the wearer's account as a “very low data” episode meaning near-zero or pico-amp data. Data recorded after a very low data episode is flagged as important for comparison by the software platform and is available to display and wearable biosensor's smart mobile device. If the electrochemical signature or other biometric markers herein change afterwards, then the system into which the invention is incorporated will detect changes in the wearer's information. A very low data signal episode is not necessary for generating comparisons of an initial biometric signature to a subsequent biometric, as described herein.

Electrochemical Signature. Detection and recognition of an electrochemical signature, either by itself or combined with other biometric techniques, create a robust and accurate identification system, thus reducing the likelihood of false positives or false negatives. The electrochemical sensor continuously samples interstitial fluid, capturing data on biochemical markers such as glucose, lactate, cortisol, alcohol, and other biochemical and/or analytes, and the wearable sensor device has biometric and/or biophysical sensors for temperature, heart rate and its variabilities, oxygen saturation levels and its variabilities, which are indicative of the user's physiological state and unique lifestyle patterns.

The electrochemical sensor as a biometric ID subsystem harnesses the power of the electrochemical make up of a person. For example, the profiling of individual-specific biomolecular biometrics from ISF or blood can verify identity using any of the following strategies: (1) DNA for individual-specific cell-free DNA sequences using functionalized electrodes for selective hybridization and redox-mediated detection; (2) RNA for individual-specific RNA patterns by employing capture probes on the sensor surface; (3) Proteins for individual-specific protein signatures using affinity-based biosensors with functionalized electrodes and a range of electrochemical detection methods; (4) Metabolites for individual-specific metabolite profiles using selective recognition elements, such as enzymes or molecularly imprinted polymers, and employing suitable electrochemical detection techniques.

The biometric ID subsystem for an electrochemical signature herein may be used in a single-analyte or a multiplex-analyte application. For this embodiment, the wearable sensor device must be capable of being configured to report target analyte levels accurately in a continuous or substantially continuous way, with substantially continuous meaning that analyte values are reported in intervals which are short enough that moving the sensor from the original wearer to a second wearer to escape detection is difficult practically.

The electrochemical sensor herein comprises, as described elsewhere herein, microneedles 2 for monitoring of analytes to create an electrochemical signature of the initial wearer. Under the supervision of the clinician, or other administrator, the wearable device is placed in the skin of the wearer so that an initial electrochemical signature may be recorded and established by the wearable biosensor. This initial electrochemical signature is then used to verify that the same wearer continues to wear the wearable biosensor. Incorporation of the initial electrochemical signature into the multi-modal ID herein provides a secure, reliable, and user-specific means of authentication, significantly reducing the possibility of identity fraud in the use of wearable devices for substance monitoring and remote clearance applications.

FIG. 2 shows aspects of data generated by one embodiment of the electrochemical sensor 2 incorporated into the wearable biosensor 1. The biosensor's connected wirelessly to an external computing device 8, here a smart mobile device running an app compatible with the wearable biosensor. The electrochemical sensor reports electrochemical data to the external computing device continuously or intermittently. The electrochemical information may include basic metabolic information and can also include information on molecules indicating use of alcohol, opioids or other compounds. Metabolic analytes in the electrochemical signature are selected from the group consisting of calcium, carbon dioxide, chloride, creatinine, glucose, potassium, sodium, urea nitrogen (BUN). As used herein sometimes, “metabolic analytes” refers to basic metabolism apart from molecules present from abuse of substances. The electrochemical signature can be established by tracking metabolic analytes or the molecules from abuse of substances. In some embodiments, a single analyte can produce an electrochemical signature and in other embodiments multiple analytes are used. In FIG. 2, at the electrochemical sensor level there is an illustration reporting sodium and potassium (Na+ and K++), cortisol (C), glucose (G), and lactate (L) over an 18 hour period for each of three days (only one day shown). The sensor also incorporates heart rate information (64 BPM), blood oxygen saturation level (94%). The software level analysis includes data fusion, machine learning, pattern recognition and identification of sensor information. The electrochemical signature subsystem is designed to profile individual-specific biomolecular metrics that are characteristic and repeatable for each user, creating a unique electrochemical signature.

Each working electrode in the array (described further in the '283 application) can be coupled with a highly sensitive electrochemical sensor capable of detecting specific analytes. These electrochemical sensors can be multiplexed to enable the simultaneous monitoring of multiple analytes, providing a comprehensive profile of the user's biochemical state. The electrochemical signature ensures another level of security by confirming the sensor's physical presence and contact with the wearer's skin. If the electrochemical sensor's status shows frequent disruptions or an unusual pattern, it might suggest tampering or improper use of the sensor.

The electrochemical signature may be used by itself or may be used in conjunction with other biometric ID subsystems for multi-modal ID. If the analyzed electrochemical pattern deviates significantly from the established signature, the system flags a potential identity mismatch.

To ensure the protection of sensitive biometric data, the electrochemical signature subsystem utilizes advanced encryption and secure data storage practices. Only anonymized, encrypted signatures are used for user verification, preserving privacy while ensuring high-security standards.

The electrochemical subsystem is designed to adapt to gradual physiological changes in the user's biomarkers due to aging, health status changes, or lifestyle modifications. Periodic calibration ensures the integrity and accuracy of the electrochemical signature over time.

By tracking metabolic analytes, the subsystem is able to detect subtle differences in the person, that is, how the person's metabolism works. These differences can include, for each of the analytes: the basic level, the highest value, a pattern of the analyte over time, a slope of increase, a slope of decline and the area under the curve. Basic level can be defined, for example, as the value present 90% of the time (e.g., fasting glucose).

From the provided information and understanding the graphs attached, a system can be created to monitor the biochemical markers of an individual over multiple days to establish a unique physiological profile, i.e. an electrochemical signature. Key aspects of this profile include fasting glucose “range”, the “ranges” of glucose accumulation and clearance rates, and glucose spike “ranges”. For instance, person X has a fasting glucose range of 89-100 mg/dl. Such a range is relatively stable and wouldn't vary drastically over a short period (e.g., day, or weeks, or months), much like one's weight or resting heart rate. If person X's sensor data showed a sudden shift to a fasting glucose range from the originally established 89-100 mg/dl to 70-80 mg/dl, this would raise a red flag, suggesting that either the sensor is not functioning correctly, or it may have been tampered with or transferred to a different person. Similarly, the expected glucose accumulation and clearance ranges, as well as the characteristic responses to glucose intake (spike ranges), are integral to this identification process. By observing these patterns over several days, the system can discern typical daily profiles and identify any anomalies. Similar analysis is used with different analytes representing basic metabolism, as listed herein.

One of the software's functions is to integrate this data using algorithms, which include machine learning and pattern recognition techniques. This allows the software to learn and adapt to the wearer's electrochemical signature, creating a more precise identification system as it collects more data over time.

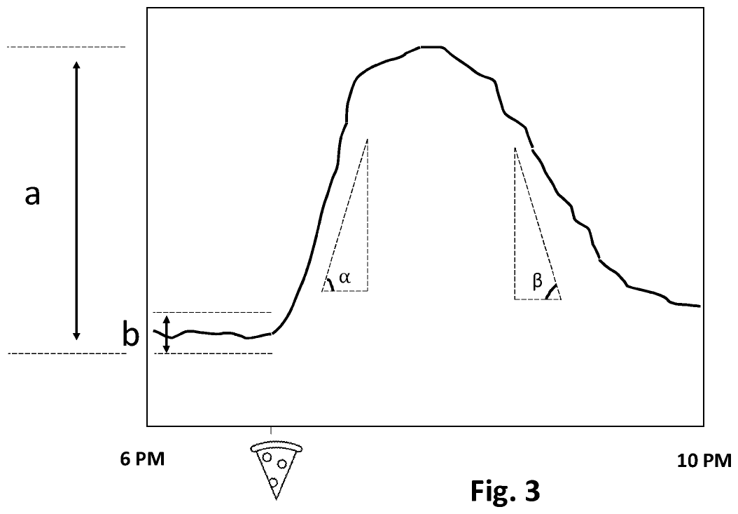

By establishing and monitoring the specific ranges of target analytes such as fasting glucose, accumulation and clearance rates, and spike responses, the continuous monitoring capability of the invention confirms the identity of the wearer over an extended period. Any significant deviations from the ranges initially established would indicate a change in the wearer's identity or a potential issue with sensor integrity. The sensor IN or OUT (i.e., very low data) as well as the embedded continuous temperature sensor are adding to the accuracy of the ID recognition system and in addition to the above-mentioned parameters. FIG. 3 is a diagram of an electrochemical signature for glucose as the target analyte. The spike range is shown by the two sided arrow labeled a. The range for fasting glucose is shown by the two sided arrow labeled b. After the “pizza” icon the glucose accumulation rate range is shown by the slope α and glucose clearance rate range is shown by the slope β.

Skin Temperature. A temperature sensor, in several different embodiments, will report important metabolic information to the wearable sensor and the companion app. Continuous monitoring of skin temperature provides data on the circadian rhythm and can also indicate changes in ambient temperature, physical activity, stress response, and latent illness—all personal characteristics that contribute to a unique temperature profile and daily patterns. At-rest skin temperature indoors will have a baseline which can be defined as the value for 90% of the time. Suitable temperature sensors include the digital Analog Devices ADT7420/ADT7320 with high precision (±0.25° C.) over a wide temperature range and the Maxim Integrated MAX30205 designed specifically for wearable and medical applications.

Heart Rate and Blood Oxygen. Heart rate and blood oxygen saturation (SpO2) sensors (e.g., MAX86140/MAX86141, Analog Devices) are examples of ultra-low-power, fully integrated, optical pulse oximeter and heart-rate sensors. These metabolic measurements serve as crucial biomarkers including evaluating cardiovascular health and determining levels of aerobic fitness, which vary from person to person and can indicate stress, activity intensity, and overall health. Together, these sensors not only reinforce the physiological profile created by the biochemical markers but also bring in the dimension of physical and locational behaviors, turning the sensor system into a highly sophisticated personal identification and health monitoring apparatus.

Glucose. This molecule is basic to life most forms of life as being a part of the chain of molecules used in energy production. It plays a big role in health and fitness and of course is important to treatment for diabetes.

Lactate. Lactate is a byproduct of anaerobic metabolism, and its levels increase after intense physical activity. Monitoring lactate over time could provide insight into the individual's exercise habits and recovery profile, which are unique to each person. Similar to glucose, resting lactate range, accumulation and clearance rates ranges, and spiking range are all individual-specific. Lactate is also an early indicator of infection.

Cortisol. Cortisol is a hormone associated with stress and the body's circadian rhythm. Its levels can indicate stress patterns, sleep cycles, and general well-being. Also, the ranges here are individual-specific.

Sodium, Potassium, Chloride, Carbon Dioxide. These electrolytes 1 are vital for bodily functions, and their levels can reflect dietary patterns, hydration status, and renal health. Also, the ranges here are individual-specific.

Creatinine and Urea Nitrogen (BUN). These are important for determining kidney function.

Calcium. This is one of the most important minerals, and is essential for the functioning of nerves, muscles and the heart.

Overall, the system's confidence in identifying an individual and detecting tamper attempts improves with each additional parameter monitored. The more physiological and behavioral data the system has, the more accurate it becomes in distinguishing between different individuals, making it a powerful tool for continuous, non-invasive ID verification.

Alcohol. The pattern of alcohol in the bloodstream can be distinctive, especially when considering habitual use. The variability in alcohol metabolism due to genetic factors, tolerance, and consumption habits adds another layer to the individual's physiological profile. Also, the ranges here are individual-specific.

Fingerprint. A fingerprint authentication subsystem of the invention is configured to capture the wearer's fingerprint as an initial biometric signature, and compare this with later fingerprint signatures, and generate a comparison. A fingerprint sensor integrated into the wearable device captures high-resolution images of the wearer's fingerprint. It employs capacitive (or optical) sensing technology to detect the unique patterns formed by the ridges and valleys on the user's fingertip. Once the fingerprint image is captured, it is converted into a digital format and passed through a series of image processing algorithms to enhance and extract distinct features. A unique identifier, known as an initial fingerprint signature, is generated from these features. This initial signature is then compared later fingerprint signatures, either locally or on a remote server, and a comparison is generated, indicating a match or not.

Gait. Another embodiment of the present invention incorporates a gait analysis subsystem configured to analyze the user's unique walking patterns and compare them with stored gait patterns, and determine a match. These may be implemented one or more of the following devices. A gyroscope such as the STMicroelectronics L3GD20H/LSM9DS1 provides stability and accuracy for motion detection, with low power consumption and sensitivity. Another gyroscope such as the InvenSense MPU-6050/MPU-9250 has a 6-axis motion tracking device (3-axis gyroscope and 3-axis accelerometer), also having a 3-axis magnetometer. The latter device thus provides a 9-axis motion tracking device which enables complex motion analysis of the wearer's gait and other movements.

The gait analysis subsystem analyzes the user's unique movement patterns in an initial gate signature, and then compares them with later gait signatures for user authentication. By leveraging this biometric modality, the wearable device ensures the device is being used on the person intended. The gait analysis subsystem collects data from various sensors embedded in the wearable device. These sensors may be selected from the group consisting of accelerometers, gyroscopes, and magnetometers that capture the user's movements in three-dimensional space. The collected data is processed to extract features representing the user's distinct gait characteristics. Feature extraction techniques, such as wavelet analysis or machine learning algorithms, can be employed to identify specific gait patterns like step length, cadence, and walking speed. These features are then combined to create a unique gait signature for each user, which is stored in a secure database for later comparison. During the authentication process, the gait analysis subsystem continuously monitors the user's walking patterns and compares the real-time gait signature with the stored gait patterns. If the system determines a match, the user is authenticated, and the wearable device functions as intended. To optimize the performance of the gait analysis subsystem, the following measures can be considered: (1) Adaptive Learning: The gait analysis subsystem may also incorporate adaptive learning algorithms that account for natural variations in a user's walking patterns over time. This can help maintain accurate identification even as the user's gait changes due to aging, injury, or other factors. The system is configured to cope with different environmental conditions, such as changes in flooring or walking surfaces, that might affect the user's gait. Signal processing techniques and machine learning algorithms are used to filter out noise and maintain accurate identification.

The GPS data provides context on geographical movement patterns, while the gyroscope and accelerometer can distinguish individual walking patterns, which are as unique as fingerprints. A GPS module such as the u-blox M8 series has high sensitivity and low power consumption, supporting multiple GNSS for improved accuracy and reliability in wearable applications where precise location tracking is required. A GPS-integrated platform such as the Qualcomm Snapdragon Wear (2100, 3100, etc.) includes integrated GPS functionality along with other sensors and computing resources. Adding GPS for location tracking, a gyroscope and accelerometer for movement and gait analysis, along with heart rate and oxygen saturation measurements, completes a comprehensive suite for personal identification and behavioral analysis.

Voice. In some embodiments, a voice authentication subsystem receives the sound of the wearer's voice in the initial identity session to process the user's voice input and extract voice features, and then compare the extracted voice features with later acquired voice data, and determines a match. The present invention's voice ID system requires a small microphone, such as those in the Knowles Sisonic™ Series, high-performance MEMS microphones that offer excellent sound quality and sensitivity and also can provide noise cancellation and directional audio capture. Other options may be found in the Infineon XENSIV™ MEMS Microphones which feature high Signal-to-Noise Ratio even in noisy environments. They provide high-quality voice input and audio recording.

Optical Recognition. At least three optical-based biometric applications may be used in different embodiments, a facial recognition subsystem may also be configured to capture the user's facial features, or an iris authentication subsystem. Each may be configured to capture the user's facial, retinal or iris features respectively using a micro-sized camera and compare them with stored facial, retinal or iris feature obtained during the initial placement by a clinician or other administrator. Later, the wearer is notified through the companion app to place his or her face or eye near the camera, and the software platform determines whether there is a match. Examples of cameras suitable for incorporation into the wearable device are as follows. The Omni Vision Technologies CameraCubeChip™ integrates a camera sensor, signal processor, and lens for applications where space and power consumption are limited, such as the present invention. Another option is the Himax Technologies HM01B0, an ultra-low power CMOS image sensor with the capability of running machine vision tasks directly on the sensor.

Data Analysis. The present invention also incorporates an authentication system configured to combine the data from all subsystems to create a reliable multi-modal ID and to grant or deny access to the wearable sensor's functions and data based on the multi-modal authentication.

The system incorporates AI and machine learning algorithms to analyze the collected biochemical data, learn the wearer-specific patterns, and establish and manipulate a biometric ID or multi-modal ID.

The AI algorithms aggregate and interpret a vast array of biometric data (including biochemical data), establishing a unique biometric signature (including electrochemical signature) through continuous learning and pattern recognition. The system uses these insights to build a dynamic, wearer-specific profile that evolves in response to physiological changes. With machine learning techniques, the system not only enhances wearer verification but also anticipates and adapts to fluctuations in biomarkers, ensuring robust and forward-looking identification protocols. This proactive approach to biometric authentication encapsulates the versatility and depth of modern AI applications in personal identification technology.

At the machine learning level, the system employs algorithms for feature extraction, utilizing techniques such as principal component analysis (PCA) to distill high-dimensional data into recognizable patterns. These patterns are then fed into predictive models—like support vector machines (SVMs) or neural networks-that undergo supervised learning with wearer-specific data to enhance the accuracy of the electrochemical (or other biometric) signature.

Using AI capability, reinforcement learning paradigms are integrated to allow the system to iteratively optimize its decision-making process, by rewarding accuracy improvements in real-time authentication scenarios. This hybrid AI strategy enables the system to dynamically adjust to the biochemical individuality of the user, improving predictive analytics and ensuring that the ID recognition system remains resilient against anomalies and potential spoofing attempts. This integration of AI fortifies the authentication system, ensuring a high degree of precision in distinguishing the rightful user's biochemical identity, thus providing an additional layer of security to the multi-modal ID recognition system.

The present invention in some embodiments comprises a communication module within the wearable device. The communications module is configured to exchange data with one or more external devices to provide timely intervention and support in the user's addiction management process, or for assistance with other uses of the wearable device into which the invention is incorporated.

The invention requires access to a power source for providing power to the continuous substance monitoring wearable device, which may include a battery, energy harvesting technologies, or other suitable means of supplying energy. In various embodiments, the invention is connected to the power source of the device into which it is incorporated.

In some embodiments an output interface, e.g. an external LCD display, is configured to provide feedback to the user regarding authentication status, sensor data, and other relevant information, such as substance levels, trends, and alerts for detected substance use or potential tampering attempts.

A data processing unit is required to analyze the data collected by the substance monitoring module and the multi-modal ID recognition system, and to manage the overall operation of the wearable device and the present invention. The electronics unit comprises the data processing unit in communication with a signal processing circuit, the data processing unit comprising a processor and a memory and configured to process the electrical signal as data representative of one or more parameters of the target analyte, and the signal processing circuit is configured to process the electrical signal by one or more of amplifying the electrical signal, filtering the electrical signal, or converting the electrical signal from analog to digital, and the data processing unit is configured to process the electrical signal after processing by the signal processing circuit. The electronics unit further comprises a wireless communication unit in communication with one or both of the signal processing circuit and the data processing unit, the wireless communication unit comprising a wireless transmitter or wireless transceiver to at least transmit one or both of the electrical signal and the data to an external computing device.

Various components and subsystems may be housed in a wearable device itself, or may be housed in an external computing device in communication with the wearable device.

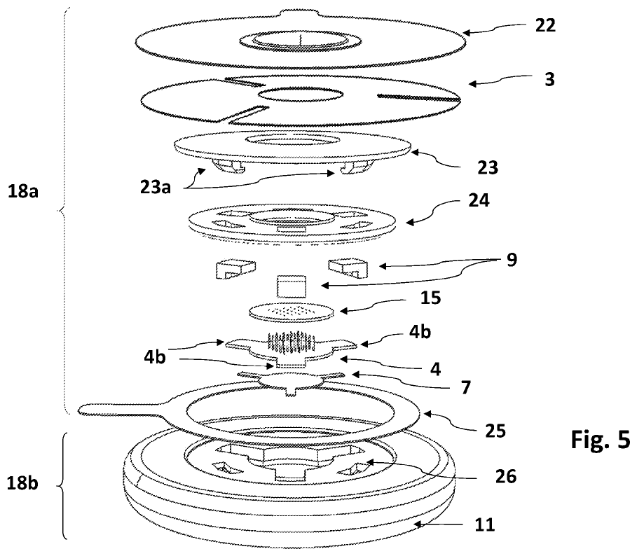

An exploded view of a fully disposable embodiment of the invention is shown in FIG. 4 (FIG. 2 of the '283 patent application with the reference numbers therefrom) and, as further described in this application comprises a recessed cover 2 which connects to a lower enclosure 11, and the microneedle array 4, and the electronics unit 10 and other components are enclosed therein. This embodiment is intended to be replaceable at the end of the life of the device, that is, it is a single unit which is fully disposable. A recessed cover 2 comprises a lower surface 2a, here in the center (without sleeves), surrounded by an upper surface 2b. At a fillet 2c, shown for example in FIG. 27 of the '283 application, a rounded annular junction at the inner boundary of the upper surface, a downward slope 2d of the recessed cover descends to the lower surface. The recessed cover in this embodiment also has at least one sidewall 2e which secures and seals it in complementary fashion to a sidewall 11a of the lower enclosure 11 which has a bottom 11b. The lower surface of the recessed cover has a set of first openings 12 (holes) which are complementary to, and correspond with, microneedles 6 of the microneedle array 4. The set of first openings can be seen clearly in FIG. 28 of the '283 application. A flexible microneedle array 4 comprises in its center a substrate 4a and microneedles 6 which at their bases 16 are integral with the substrate. In the embodiment of FIG. 4, a force touch sensor 7 underneath the microneedle array is shown, for example, in FIG. 5 (FIG. 3 of the '283 application). An electronics unit 10 here is either a printed circuit board (PCB) or application specific integrated circuit (ASIC). Conductive pins 9 at the cantilevered arms 4b of the microneedle array attach to substrate contacts and to contacts on the electronics unit. Connection holes 4c allow a connection to contacts 10a on the electronics unit by conductive pins 9 which may be flexible and elastic or rigid. A skin adhesive layer 3 is attached to the upper surface of the recessed cover, that is, there is an opening in the skin adhesive layer 3 above the microneedles as they would appear through the first openings in the lower surface. The skin adhesive layer may have slots 3a to prevent wrinkling.

An embodiment which is partially reusable is shown in FIG. 5. Here the disposable module 18a comprising the microneedle array can be separated after usage for the recommended period and a replacement disposable module can be affixed to the reusable module 18b. FIG. 3 is an exploded perspective view of the two modules 18a, 18b of the device with a removable microneedle protector 22, a skin adhesive layer 3, a rigid base 23, an optional elastic insulative base 24, pins 9 (here, compression pins in an L-shape), a basic cover 15, a microneedle array 4 with microneedles 6 and cantilevered arms 4b, a force touch sensor 7. All the above is in module 18a and removable with an eject plastic sheet 25. This disposable module sits above the reusable module 18b with a lower rigid base 26 with an electronics unit (enclosed but not visible) inside a lower enclosure 11.

FIG. 6A (FIG. 4A of the '283 application) is similar to FIG. 5, except that a recessed cover 2 (without a sidewall) is substituted for the basic cover 15. An elastic insulative base 24 is present to give greater flexibility and conformability to the microneedle array, for better contact and retention on the skin without movement. FIG. 6A is similar to FIG. 6B (FIG. 4B of the '283 application) except that the latter has no elastic insulative base. FIG. 5A of the '283 application) is a perspective view of one embodiment of the wearable biosensor device 1 as a two module device, with the replaceable module 18a in FIG. 5B of the '283 application and the reusable module 18b in FIG. 5C of the '283 application.

### Additional Examples

The invention has many other embodiments besides those disclosed above, including examples 1-12 and others contained herein.

Example 1 is a system for verifying the identity of a wearer of a wearable biosensor device, the system comprising a wearable biosensor device comprising a microneedle array comprising a substrate integral with microneedles configured for placement within skin of the wearer, a biorecognition element on a portion of the microneedles configured to react with at least one target analyte in the skin, and electrical connections between the biorecognition element and an electronics unit for data processing, and the system further comprises at least one biometric ID subsystem configured to establish an initial biometric signature of the wearer, to store the initial biometric signature in a database, and to generate a comparison of the initial biometric signature to at least one later biometric signature.

Example 2 includes the device of any of examples 1-12 wherein the at least one biometric ID subsystem comprises a data fusion module, a pattern recognition module, a machine learning module, and an authentication module.

Example 3 includes the device of any of examples 1-12 wherein the at least one biometric ID subsystem is selected from the group consisting of a subsystem for recognition of the wearer by electrochemical signature, voice, face, iris, retina, fingerprint, and gait.

Example 4 includes the device of any of examples 1-12 wherein the subsystem for recognition by electrochemical signature comprises a set of electrochemical values for the target analyte of the wearer selected from the group consisting of a baseline level, accumulation and clearance rates, spike responses and area under the curve.

Example 5 includes the device of any of examples 1-12 wherein the electrochemical signature is derived for one or more of the target analytes.

Example 6 includes the device of any of examples 1-12 wherein the subsystem for recognition by gait comprises a GPS device for location in the wearable biosensor device.

Example 7 includes the device of any of examples 1-12 wherein the at least one biometric ID system is a multi-modal ID system comprising at least two of the systems for recognition of the wearer.

Example 8 includes the device of any of examples 1-12 wherein the system is configured to generate the comparison of the initial biometric signature to the at least one later biometric signature during or shortly after a very low data episode.

Example 9 includes the device of any of examples 1-12 wherein the system is configured to generate the comparison of the initial biometric signature to the at least one later biometric signature spontaneously or at random times.

Example 10 includes the device of any of examples 1-12 wherein the system is configured to generate the comparison of the initial biometric signature to the at least one later biometric signature at intervals prompted by the pattern recognition module.

Example 11 includes the device of any of examples 1-12 wherein the electronics unit comprises a data processing unit in communication with a signal processing circuit, the data processing unit comprising a processor and a memory and configured to process the electrical signal as data representative of one or more parameters of the target analyte.

Example 12 includes the device of any of examples 1-12 wherein the signal processing circuit is configured to process the electrical signal by one or more of amplifying the electrical signal, filtering the electrical signal, or converting the electrical signal from analog to digital, and wherein the data processing unit is configured to process the electrical signal after processing by the signal processing circuit.

Example 13 includes the device of any of examples 1-12 wherein the electronics unit further comprises a wireless communication unit in communication with one or both of the signal processing circuit and the data processing unit, the wireless communication unit comprising a wireless transmitter or wireless transceiver to at least transmit one or both of the electrical signal and the data to an external computing device.

1. A system for verifying the identity of a wearer of a wearable biosensor device, the system comprising a wearable biosensor device comprising a microneedle array comprising a substrate integral with microneedles configured for placement within skin of the wearer, a biorecognition element on a portion of the microneedles configured to react with at least one target analyte in the skin, and electrical connections between the biorecognition element and an electronics unit for data processing, and the system further comprises at least one biometric ID subsystem configured to establish an initial biometric signature of the wearer, to store the initial biometric signature in a database, and to generate a comparison of the initial biometric signature to at least one later biometric signature.

2. The system as in claim 1 wherein the at least one biometric ID subsystem comprises a data fusion module, a pattern recognition module, a machine learning module, and an authentication module.

3. The system as in claim 1 wherein the at least one biometric ID subsystem is selected from the group consisting of a subsystem for recognition of the wearer by electrochemical signature, voice, face, iris, retina, fingerprint, and gait.

4. The system as in claim 3 wherein the subsystem for recognition by electrochemical signature comprises a set of electrochemical values for the target analyte of the wearer selected from the group consisting of a baseline level, accumulation and clearance rates, spike responses and area under the curve.

5. The system as in claim 4 wherein the electrochemical signature is derived for one or more of the target analytes.

6. The system as in claim 3 wherein the subsystem for recognition by gait comprises a GPS device for location in the wearable biosensor device.

7. The system as in claim 3 wherein the at least one biometric ID system is a multi-modal ID system comprising at least two of the systems for recognition of the wearer.

8. The system as in claim 1 wherein the system is configured to generate the comparison of the initial biometric signature to the at least one later biometric signature during or shortly after a very low data episode.

9. The system as in claim 1 wherein the system is configured to generate the comparison of the initial biometric signature to the at least one later biometric signature spontaneously or at random times.

10. The system as in claim 1 wherein the system is configured to generate the comparison of the initial biometric signature to the at least one later biometric signature at intervals prompted by the pattern recognition module.

11. The device of claim 1, wherein the electronics unit comprises a data processing unit in communication with a signal processing circuit, the data processing unit comprising a processor and a memory and configured to process the electrical signal as data representative of one or more parameters of the target analyte.

12. The device of claim 11, wherein the signal processing circuit is configured to process the electrical signal by one or more of amplifying the electrical signal, filtering the electrical signal, or converting the electrical signal from analog to digital, and wherein the data processing unit is configured to process the electrical signal after processing by the signal processing circuit.

13. The device of claim 12, wherein the electronics unit further comprises a wireless communication unit in communication with one or both of the signal processing circuit and the data processing unit, the wireless communication unit comprising a wireless transmitter or wireless transceiver to at least transmit one or both of the electrical signal and the data to an external computing device.
